(19) United States
(12) Patent Application Publication
(10) Pub. No.: US 2024/0355479 A1
(43) Pub. Date: Oct. 24, 2024

(54) MEASUREMENT SYSTEM, MEASUREMENT APPARATUS, MEASUREMENT DATA PROCESSING METHOD, AND MEASUREMENT DATA PROCESSING PROGRAM

(71) Applicant: HITACHI HIGH-TECH CORPORATION, Tokyo (JP)

(72) Inventors: Hiromitsu NAKAGAWA, Tokyo (JP); Takeshi TANAKA, Tokyo (JP); Daisuke FUKUI, Tokyo (JP)

(73) Assignee: HITACHI HIGH-TECH CORPORATION, Tokyo (JP)

(21) Appl. No.: 18/580,973

(22) PCT Filed: Jan. 24, 2022
(86) PCT No.: PCT/JP2022/002448

§ 371 Date: Jan. 19, 2024

(30) Foreign Application Priority Data
Aug. 2, 2021 (JP) 2021-126860

(51) Int. Cl. G16H 50/30; G06T 7/00; G06T 7/70; G06V 40/20; G16H 30/40

(52) U.S. Cl. CPC G16H 50/30; G06T 7/0016; G06T 7/70; G06V 40/25; G16H 30/40; G06T 2207/20081; G06V 2201/033

(57) ABSTRACT

A measurement apparatus includes: a measurement data acquisition unit configured to acquire measurement data of a target; and a processing unit configured to process the measurement data. The processing unit estimates a skeleton of a living body that is the target based on the measurement data, estimates a measurement condition related to the measurement data using an estimation result of the skeleton, obtains a similarity between the measurement condition and an assumed condition assumed in advance, and selects measurement data related to evaluation of a state of the target based on the similarity. With the configuration and operations,, it is possible to efficiently collect data related to evaluation of a state of a target while considering a restriction of a location where measurement data is acquired, without collecting a large amount of data.

### TECHNICAL FIELD

The present invention relates to a measurement system, a measurement apparatus, a measurement data processing method, and a measurement data processing program.

### BACKGROUND ART

In recent care industry has been attracting attention due to an increase in health awareness of people who have taken into account the future of a super-ageing society. Along with this, technology application of information technology (IT) in the medical field has also been advanced, and new business fields and medical services such as regenerative medical equipment, nursing care robots, and insurance guidance service support have been developed one after another.

Among them, a preventive medical service is one of the fields which are highly expected in view of ageing-dedicated medical care. The preventive medicine is not a method of undergoing treatment after getting sick, but is a method of clarifying a health risk that may arise in the future for anyone and continuously performing health maintenance and enhancement actions so as not to get sick.

In order to clarify the health risk and confirm the effects of health maintenance and enhancement actions, it is necessary to continuously quantify a health state by measurement. With the progress of Internet of things (IOT) technology and wearable devices, each user can perform measurement in daily life for quantification.

On the other hand, regarding measurement data obtained in daily life, a measurement condition thereof may vary and it may be difficult to perform highly reliable analysis. For example, in a case of imaging a walking state to be used for analysis, when there is a difference in conditions that a certain user images a walking state from the front and that another user images a walking state from the side, it is not possible to simply compare the walking of both users.

In order to obtain data contributing to analysis, there is a method of collecting a large amount of data. For example, PTL 1 discloses that feature dictionary data is created from camera videos comprehensively collected at a plurality of sites and heterogeneous data. Accordingly, highly reliable analysis can be implemented.

It is also conceivable to control acquisition conditions to efficiently acquire data contributing to analysis. For example, PTL 2 discloses that training data is analyzed and a camera is controlled so that test data is imaged under the same imaging condition. Accordingly, highly reliable analysis can be implemented.

### CITATION LIST

### Patent Literature

PTL 1: JP2014-59729A

PTL 2: WO2018/100676

### SUMMARY OF INVENTION

### Technical Problem

There is a problem in the technique disclosed in PTL 1 that, although the highly reliable analysis can be performed, it is costly to collect a large amount of data.

In the technique disclosed in PTL 2, there is a problem that, although the highly reliable analysis can be performed, a restriction of an imaging location on camera control is not considered.

For this reason, it is an important issue to efficiently collect data related to evaluation of a state of a target while considering the restriction of an imaging location, without collecting a large amount of data.

### Solution to Problem

In order to achieve the above object, a representative measurement system and a representative measurement apparatus of the invention include: a measurement data acquisition unit configured to acquire measurement data of a target; and a processing unit configured to process the measurement data. The processing unit estimates a skeleton of a living body that is the target based on the measurement data, estimates a measurement condition related to the measurement data using an estimation result of the skeleton, obtains a similarity between the measurement condition and an assumed condition assumed in advance, and selects measurement data related to evaluation of a state of the target based on the similarity.

A representative measurement data processing method according to the invention includes: a measurement data acquisition step of acquiring measurement data of a target; a skeleton estimation step of estimating a skeleton of a living body that is the target based on the measurement data; a condition estimation step of estimating a measurement condition related to the measurement data using an estimation result of the skeleton; a similarity calculation step of obtaining a similarity between the measurement condition and an assumed condition assumed in advance; and a selecting step of selecting measurement data related to evaluation of a state of the target based on the similarity.

A representative measurement data processing program according to the invention causes a computer to execute: a process of receiving measurement data of a target; a process of estimating a skeleton of a living body that is the target based on the measurement data; a process of estimating a measurement condition related to the measurement data using an estimation result of the skeleton; a process of obtaining a similarity between the measurement condition and an assumed condition assumed in advance; and a process of selecting measurement data related to evaluation of a state of the target based on the similarity.

### Advantageous Effects of Invention

According to the invention, it is possible to efficiently collect data related to evaluation of a state of a target. Problems, configurations, and effects other than those described above will become apparent in the following description of embodiments.

### BRIEF DESCRIPTION OF DRAWINGS

FIG. 1 is a physical configuration diagram of an entire system according to a first embodiment.

FIG. 2 is a functional configuration diagram of the entire system according to the first embodiment.

FIG. 3 is an explanatory diagram illustrating a configuration of a table stored in a measurement DB.

FIG. 4 is an explanatory diagram illustrating a configuration of a table stored in a determination DB.

FIG. 5 is an explanatory diagram illustrating a configuration of a table stored in an analysis DB.

FIG. 6 is a flowchart illustrating a depth recording process.

FIG. 7 is a flowchart illustrating a flow of a skeleton coordinate estimation process.

FIG. 8 is a flowchart illustrating a flow of a measurement condition estimation process.

FIG. 9 is a flowchart illustrating a flow of a similarity calculation process.

FIG. 10 is a flowchart illustrating a flow of an analysis target selection process.

FIG. 11 is a logical configuration diagram of an entire system according to a second embodiment.

FIG. 12 is a flowchart illustrating a flow of a determination control process.

FIG. 13 is a flowchart illustrating a flow of a measurement condition determination process.

FIG. 14 is a flowchart illustrating a flow of a U/I control process.

FIG. 15 is an explanatory diagram illustrating an example of a configuration of a measurement condition confirmation screen.

### DESCRIPTION OF EMBODIMENTS

Hereinafter, several embodiments of the invention will be described with reference to the drawings. These embodiments are merely examples for implementing the invention, and do not limit the technical scope of the invention.

In the following description, an “interface unit” is one or more interface devices. One or more interfaces may be one or more interface devices of the same type (for example, one or more network interface cards (NIC) or two or more interface devices of different types (for example, NIC and host bus adapter (HBA)).

In the following description, a “storage unit” is one or more memories. At least one memory may be a volatile memory or a nonvolatile memory. The storage unit may include one or more PDEVs in addition to one or more memories. The “PDEV” refers to a physical storage device, and may be typically a nonvolatile storage device (for example, an auxiliary storage device). The PDEV may be, for example, a hard disk drive (HDD) or a solid state drive (SSD).

In the following description, a “processor unit” is one or more processors. At least one processor is typically a central processing unit (CPU). The processor may include a hardware circuit that performs some of or all of the processes.

In the following description, a function may be indicated by the expression of “kkk unit” (excluding the interface unit, the storage unit, and the processor unit), and the function may be implemented by the processor unit executing one or more computer programs, or may be implemented by one or more hardware circuits (for example, a field-programmable gate array (FPGA) or an application-specific integrated circuit (ASIC)). When the function is implemented by the processor unit executing a program, a predetermined process is performed using the storage unit and/or the interface unit as appropriate, and thus the function may be at least a part of the processor unit. A process described with the function as a subject may be a process performed by the processor unit or a device including the processor unit. The program may be installed from a program source. The program source may be, for example, a program distribution computer or a computer-readable recording medium (for example, a non-transitory recording medium). A description of each function is an example. A plurality of functions may be integrated into one function, or one function may be divided into a plurality of functions.

In the following description, information may be described by an expression of “xxx table”, and the information may be expressed in any data structure. That is, in order to indicate that the information does not depend on the data structure, the “xxx table” can be referred to as “xxx information”. In the following description, a configuration of each table is an example. One table may be divided into two or more tables, or all or some of two or more tables may be one table.

In the following description, “time” is expressed in units of year, month, day, hour, minute, and second. Alternatively, the unit of time may be rougher or finer than that, or may be a different unit.

In the following description, a “data set” means data (a cluster of logical electronic data) including one or more data elements, and may be any of a record, a file, a key-value pair, and a tuple, for example.

### First Embodiment

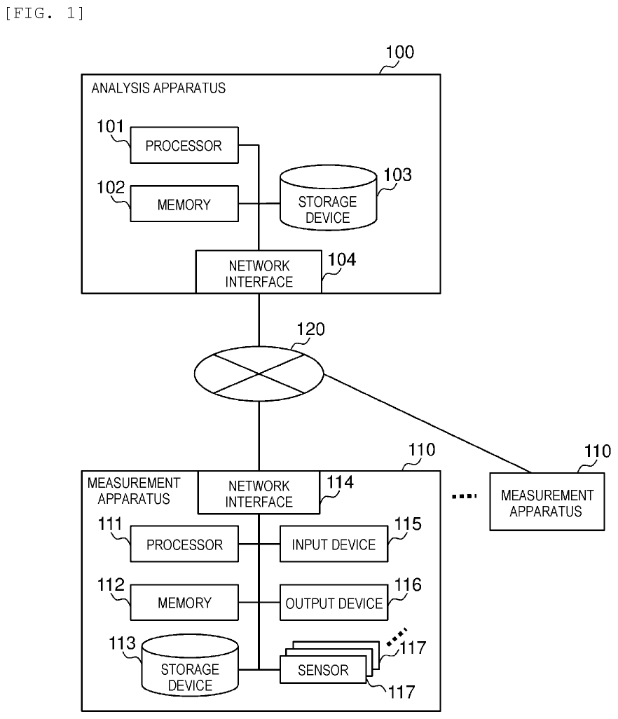

FIG. 1 illustrates a physical configuration of an entire system according to a first embodiment. A measurement system illustrated in FIG. 1 has a configuration in which an analysis apparatus 100 is connected to a plurality of measurement apparatuses 110 via a network 120.

The analysis apparatus 100 is an apparatus that analyzes data. The analysis apparatus 100 includes a network interface 104, a memory 102, a storage device 103, and a processor 101 connected thereto. The analysis apparatus 100 implements a data analysis function by loading a program stored in the storage device 103 into the memory 102 and sequentially executing the program by the processor 101.

Specifically, the analysis apparatus 100 trains a machine learning model using, as training data, a health state of a person and measurement data obtained by measuring a walking state of the person, thereby quantifying an index indicating the health state from natural walking. This index is useful for evaluating a disease or the like related to orthopedics or a cranial nervous system that affects functions of a human musculoskeletal system.

The analysis apparatus 100 collects measurement data from the plurality of measurement apparatuses 110 via the network 120 and the network interface 104. The measurement data can be used to train a machine learning model or evaluate a health state by a trained machine learning model.

The measurement apparatus 110 includes a processor 111, a memory 112, a storage device 113, a network interface 114, an input device 115, an output device 116, and sensors 117. The input device 115 is an interface configured to receive input from an operator of the measurement apparatus 110, and is, for example, an operation button, a keyboard, or a touch panel. The output device 116 is an interface that performs output to the operator, and is, for example, a display device.

The sensor 117 is a device configured to acquire measurement data. Although any sensor can be used as the sensor 117, in the embodiment, a case where an imaging device (for example, a Time-of-Flight Camera) that captures images in time series and associates depth data with each pixel is adopted as the sensor 117 will be described as an example.

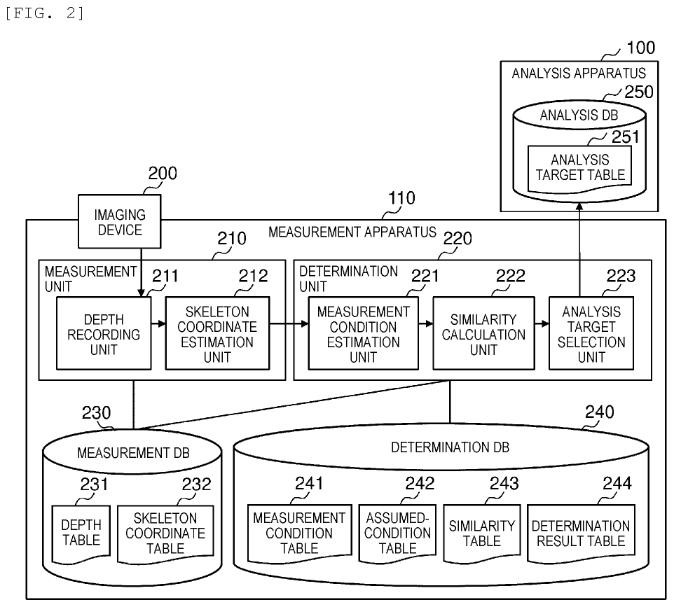

The measurement apparatus 110 implements various functions by loading a program stored in the storage device 113 into the memory 112 and sequentially executes the program by the processor 111. The functions of the measurement apparatus 110 will be described with reference to FIG. 2.

FIG. 2 is a functional configuration diagram of the entire system according to the first embodiment. As illustrated in FIG. 2, the measurement apparatus 110 includes an imaging device 200, a measurement unit 210, a determination unit 220, a measurement database (DB) 230, and a determination DB 240.

The imaging device 200 is one of the sensors 117, and acquires the time-series image data and depth data as measurement data as described above. The measurement unit 210 and the determination unit 220 are functions implemented by the processor 111 executing a program. The measurement DB 230 and the determination DB 240 are stored in the storage device 113.

The measurement unit 210 includes a depth recording unit 211 and a skeleton coordinate estimation unit 212. The depth recording unit 211 stores measurement data as a depth table 231 in the measurement DB 230. The skeleton coordinate estimation unit 212 estimates a skeleton of a living body that is the target based on the measurement data. Specifically, the skeleton coordinate estimation unit 212 refers to the depth table 231, estimates joint coordinates of a person that is a measurement target, and stores the joint coordinates as a skeleton coordinate table 232 in the measurement DB 230.

The determination unit 220 includes a measurement condition estimation unit 221, a similarity calculation unit 222, and an analysis target selection unit 223. The measurement condition estimation unit 221 estimates a measurement condition related to the measurement data by using an estimation result of the skeleton. Specifically, the measurement condition estimation unit 221 calculates a horizontal plane and a traveling direction of walking based on ground coordinates of skeleton data, and obtains measurement conditions such as a walking distance and angle based on the horizontal plane, the traveling direction, and a camera coordinate system. The measurement conditions may include an installation height and an imaging direction of the camera. The measurement condition estimation unit 221 stores the estimated measurement condition as a measurement condition table 241 in the determination DB 240.

The similarity calculation unit 222 determines a similarity between the measurement condition and an assumed condition assumed in advance, and assigns the similarity to the measurement data. Specifically, the similarity calculation unit 222 obtains the similarity using a value obtained by normalizing a difference between the measurement condition and the assumed condition, and stores the similarity as a similarity table 243 in the determination DB 240.

The analysis target selection unit 223 selects the measurement data based on the similarity. Specifically, the analysis target selection unit 223 determines for each item whether the similarity is equal to or greater than a similarity threshold, stores a determination result in a determination result table 244, selects the measurement data based on the determination result of each item, and transmits the measurement data to the analysis apparatus 100.

The assumed condition used by the similarity calculation unit 222 and the similarity threshold used by the analysis target selection unit are stored as an assumed-condition table 242 in the determination DB 240.

The analysis apparatus 100 stores data, which is received from the measurement apparatus 110, as an analysis target table 251 in an analysis DB 250. The analysis DB 250 is constructed in the storage device 103 provided in the analysis apparatus 100. The analysis apparatus 100 can use data of the analysis target table for training a learning model or evaluating a target.

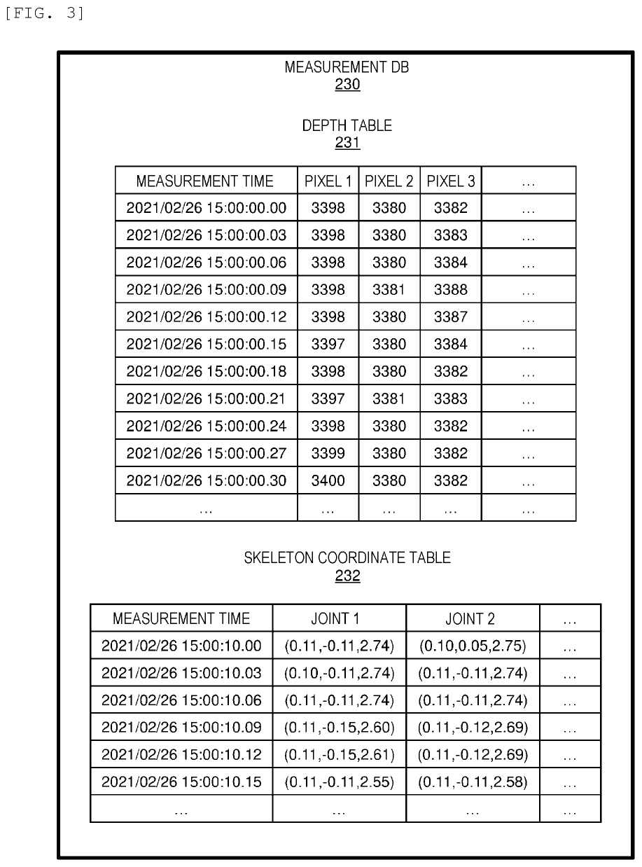

FIG. 3 is an explanatory diagram illustrating a configuration of the tables stored in the measurement DB. The depth table 231 stored in the measurement DB 230 is a table in which a measurement time is associated with a depth value of each pixel. Here, one row in the depth table 231 indicates one image acquired at a certain time, and for convenience, one row is defined as one record and is referred to as a depth record. In measurement of walking, with respect to a certain degree of time range, specifically, a time range in which a walking pattern presented by several steps or more can occur, data acquired in a time series is handled as a measurement data group of one set, and in the depth table 231 in FIG. 3, a plurality of depth records corresponding to the time range are used as the measurement data group of one set.

The skeleton coordinate table 232 stored in the measurement DB 230 is a table in which the measurement time is associated with coordinates of each joint. One row in the skeleton coordinate table 232 indicates coordinates of a plurality of joints included in one image acquired at a certain time, and for convenience, one row is defined as one record and is referred to as a skeleton coordinate record. With respect to the skeleton coordinate record, a plurality of skeleton coordinate records corresponding to the time range correspond to a measurement data group of one set.

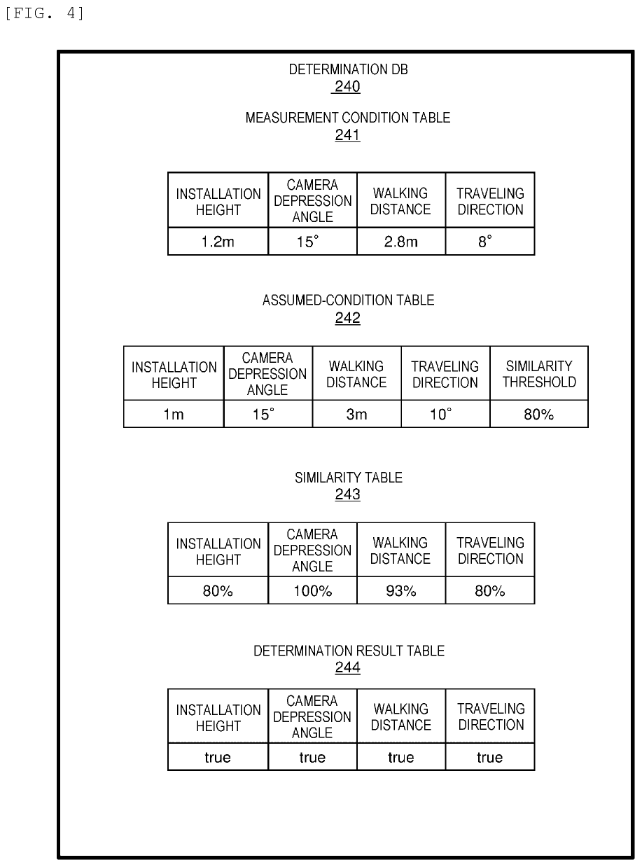

FIG. 4 is an explanatory diagram illustrating a configuration of the tables stored in the determination DB. The measurement condition table 241 stored in the determination DB 240 is a table including items of installation height, camera depression angle, walking distance, and traveling direction. The installation height indicates a height at which the imaging device 200 is installed with respect to a floor surface on which a target person walks. The camera depression angle is a height component of the imaging direction of the imaging device 200 with respect to a walking direction. The walking distance indicates a distance over which the target walked. The traveling direction is a horizontal component of the imaging direction of the imaging device 200 with respect to the walking direction. The measurement condition is obtained for the measurement data group. That is, one measurement condition is obtained from a plurality of skeleton coordinate records corresponding to a predetermined time range. For convenience, the items of the measurement condition table 241 are referred to as condition columns of the measurement condition table 241.

The assumed-condition table 242 shows items of installation height, camera depression angle, walking distance, traveling direction, and similarity threshold. Although the items of installation height, camera depression angle, walking distance, and traveling direction are similar to those of the measurement condition table 241, the measurement condition table 241 shows the conditions at the time of acquisition of the measurement data, whereas the assumed-condition table 242 indicates values assumed in advance for desirable conditions at the time of acquisition of the measurement data. The similarity threshold is used when selecting the measurement data as the analysis target data.

The similarity table 243 shows a similarity between each item (condition column) included in the measurement condition table 241 and the assumed condition shown in the assumed-condition table 242. For convenience, the items of the similarity table 243 are referred to as condition columns of the similarity table 243. The determination result table 244 stores, for each item (condition column) included in the measurement condition table 241, a value of “true” when the similarity is equal to or greater than the similarity threshold of the assumed-condition table 242, and a value of “false” when the similarity is less than the similarity threshold.

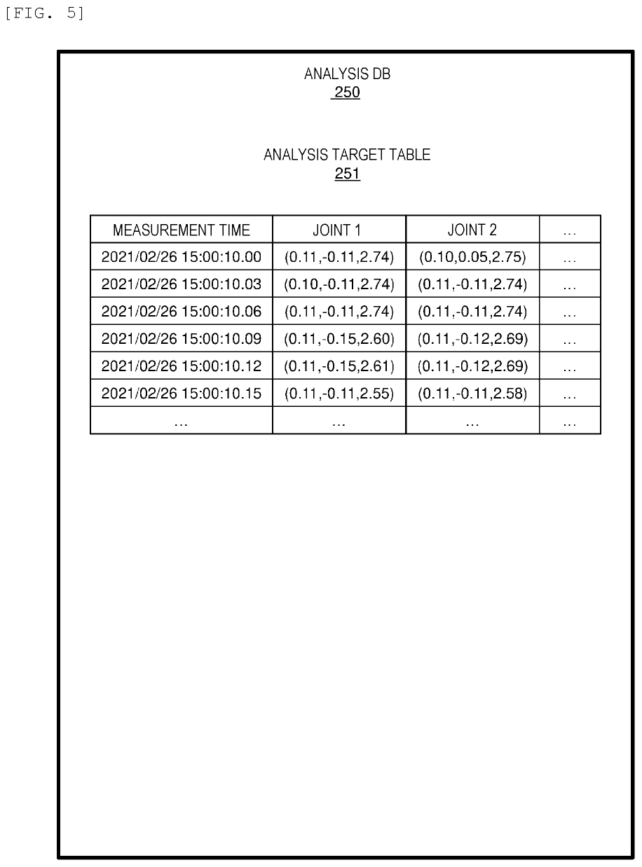

FIG. 5 is an explanatory diagram illustrating a configuration of the table stored in the analysis DB. Similarly to the skeleton coordinate table 232 stored in the measurement DB 230, the analysis target table 251 stored in the analysis DB 250 is a table in which the measurement time is associated with coordinates for each joint. In the skeleton coordinate table 232, data of skeleton estimated for each of all measurement data groups is registered. Among the data registered in the skeleton coordinate table 232, data whose measurement condition is similar to the assumed condition is selected as an analysis target and registered in the analysis target table 251. At this time, selection and registration are performed with respect to the measurement data group. That is, all of a plurality of skeleton coordinate records corresponding to one measurement data group are selected as one set and registered in the analysis target table 251.

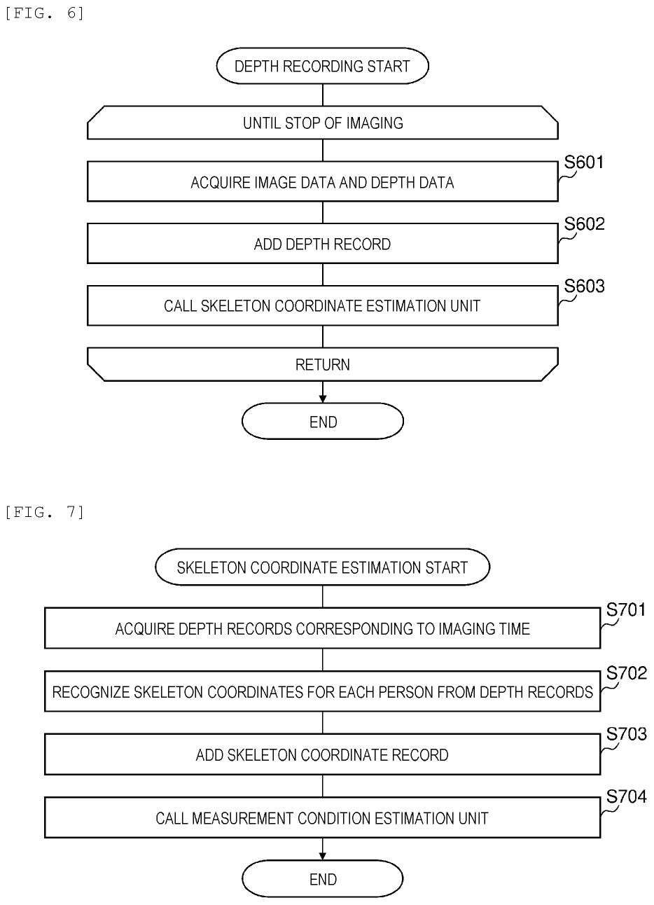

FIG. 6 is a flowchart illustrating a flow of a depth recording process. The depth recording unit 211 repeats processes of step S601 to step S603 from start to stop of imaging of the imaging device 200.

Step S601 is a step in which the depth recording unit 211 acquires image data and depth data.

Step S602 is a step in which the depth recording unit 211 registers a depth record in the depth table 231 based on the acquired image data and depth data.

Step S603 is a step in which the depth recording unit 211 calls the skeleton coordinate estimation unit 212.

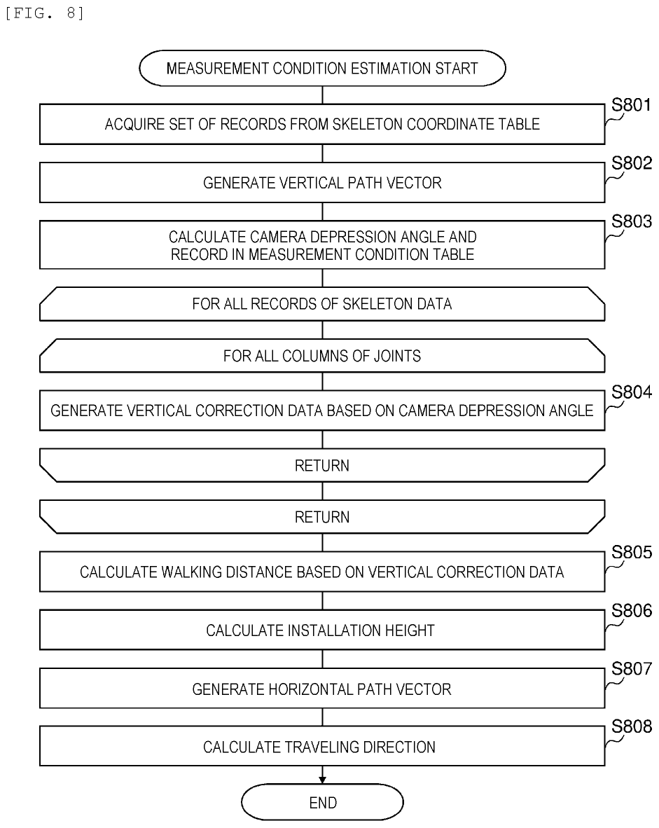

FIG. 7 is a flowchart illustrating a flow of a skeleton coordinate estimation process. The skeleton coordinate estimation unit 212 called by the depth recording unit 211 sequentially executes processes of step S701 to step S704.

Step S701 is a step in which the skeleton coordinate estimation unit 212 acquires depth records corresponding to an imaging time from the depth table 231.

Step S702 is a step in which the skeleton coordinate estimation unit 212 recognizes skeleton coordinates of each person from the depth records. Any method can be used to recognize skeleton coordinates.

Step S703 is a step in which the skeleton coordinate estimation unit 212 registers a skeleton coordinate record in the skeleton coordinate table 232.

Step S704 is a step in which the skeleton coordinate estimation unit 212 calls the measurement condition estimation unit 221.

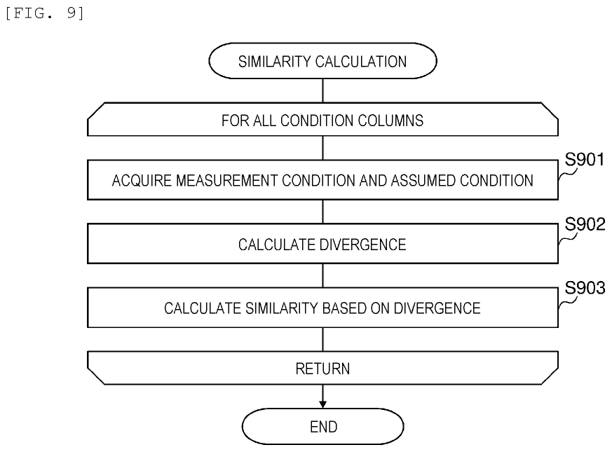

FIG. 8 is a flowchart illustrating a flow of a measurement condition estimation process. The measurement condition estimation unit 221 called by the skeleton coordinate estimation unit 212 first sequentially executes processes of step S801 to step S803.

Step S801 is a step in which the measurement condition estimation unit 221 acquires a set of records corresponding to one measurement data group from the skeleton coordinate table.

The records acquired at this time may be all the skeleton coordinate records within a time range corresponding to one measurement data group, and a first record and a last record within the time range may be selectively acquired.

Step S802 is a step in which the measurement condition estimation unit 221 generates a vertical path vector. Here, the coordinates in the skeleton coordinate table use an xyz coordinate system in which a depth direction of the image is defined as a z-axis, a longitudinal direction of the image is defined as a y-axis, and a transverse direction of the image is defined as an x-axis. The measurement condition estimation unit 221 generates the vertical path vector based on (y-coordinate, z-coordinate) of a predetermined joint of the first record and (y-coordinate, z-coordinate) of the predetermined joint of the last record.

Step S803 is a step in which the measurement condition estimation unit 221 calculates a camera depression angle and records the camera depression angle in the measurement condition table 241. The measurement condition estimation unit 221 calculates an angle formed by the vertical path vector with reference to the z-coordinate axis of the camera, and records the angle in the column of the camera depression angle of the measurement condition table.

After step S803, the measurement condition estimation unit 221 executes a process of step S804 for all joints of all records acquired in step S801.

Step S804 is a process in which the measurement condition estimation unit 221 generates vertical correction data based on the camera depression angle. The measurement condition estimation unit 221 rotates all (y-coordinates, z-coordinates) using the camera depression angle, and hold in the memory as vertical correction data. By this rotation, a z coordinate axis becomes horizontal to the floor surface, and the y coordinate axis becomes vertical to the e floor surface. The x coordinate axis is horizontal to the floor surface at the time of imaging, and does not need to be rotated.

After the process of step S804 is executed for all the joints of all the records, the measurement condition estimation unit 221 sequentially executes processes of steps S805 to S808.

Step S805 is a step in which the measurement condition estimation unit 221 calculates a walking distance based on the vertical correction data. The measurement condition estimation unit 221 calculates a difference between the maximum z-coordinate and the minimum z-coordinate of the vertical correction data, and records the difference in the column of the walking distance of the measurement condition table 241. Here, it is assumed that imaging is performed when the target person walks toward the imaging device 200. Such a state may be implemented by installing the imaging device 200 in a corridor or prompting walking along a line drawn on a floor surface.

Step S806 is a step in which the measurement condition estimation unit 221 calculates the installation height. The measurement condition estimation unit 221 calculates a difference between an origin, which is a position of the imaging device 200, and the minimum y-coordinate of the vertical correction data, and records the difference in the column of the installation height of the measurement condition table 241.

Step S807 is a step in which the measurement condition estimation unit 221 generates a horizontal path vector. The measurement condition estimation unit 221 generates the horizontal path vector from (x-coordinate, z-coordinate) of the predetermined joint of the first record and (x-coordinate, z-coordinate) of the predetermined joint of the last record.

Step S808 is a step in which the measurement condition estimation unit 221 calculates the traveling direction. The measurement condition estimation unit 221 calculates an angle formed by the horizontal path vector with reference to the x-coordinate axis of the imaging device 200, and records the angle in the column of the traveling direction of the measurement condition table 241.

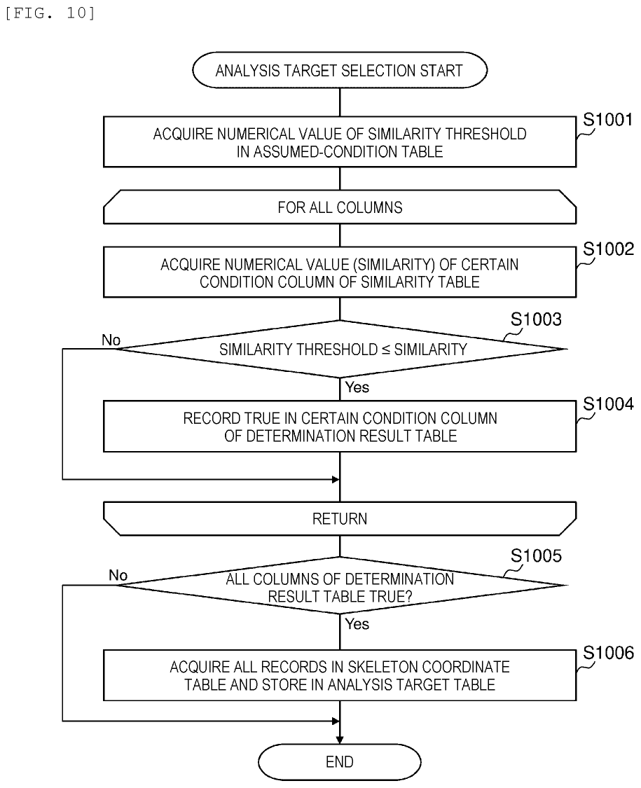

FIG. 9 is a flowchart illustrating a flow of a similarity calculation process. The similarity calculation unit 222 sequentially executes processes of step S901 to step S903 for all condition columns of the measurement condition table 241.

Step S901 is a step in which the similarity calculation unit 222 acquires the measurement condition and the assumed condition. The similarity calculation unit 222 acquires a numerical value of a certain condition column of the measurement condition table 241 and a numerical value of a certain condition column of the assumed-condition table 242.

Step S902 is a step in which the similarity calculation unit 222 calculates a discrepancy. The similarity calculation unit 222 normalizes a difference between the value of the condition column of the measurement condition table 241 and the value of the condition column of the assumed-condition table 242 to calculate the discrepancy.

Step S903 is a step in which the similarity calculation unit 222 calculates the similarity based on the discrepancy. The similarity calculation unit 222 subtracts a numerical value obtained by multiplying the discrepancy by 100 from 100 to calculate the similarity, and records the similarity in the condition column of the similarity table 243.

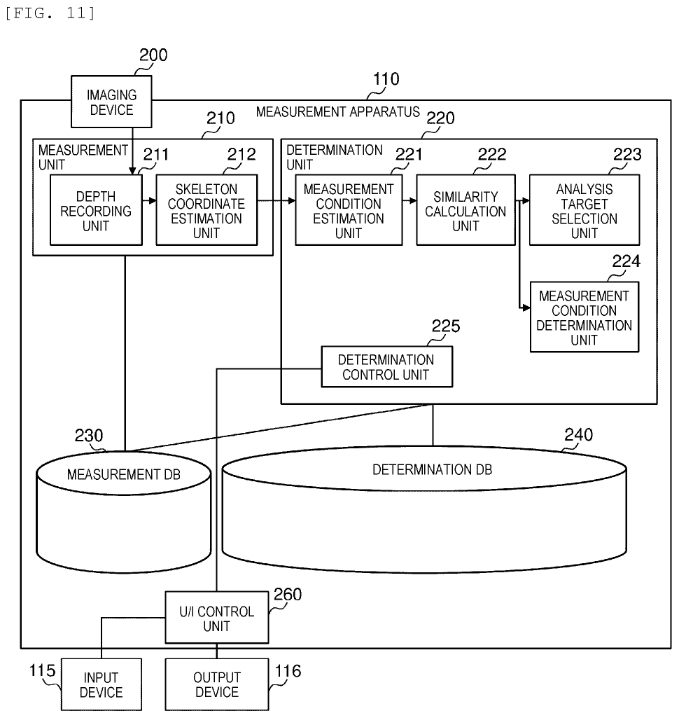

FIG. 10 is a flowchart illustrating a flow of an analysis target selection process. The analysis target selection unit 223 first executes a next step S1001.

Step S1001 is a step in which the analysis target selection unit 223 acquires a numerical value of the similarity threshold in the assumed-condition table 242. Thereafter, the analysis target selection unit 223 executes looping of step S1002 to step S1004 for all the columns.

Step S1002 is a step in which the analysis target selection unit 223 acquires a numerical value (similarity) of a certain condition column of the similarity table.

Step S1003 is a step in which the analysis target selection unit 223 compares the similarity threshold acquired in step S1001 with the similarity acquired in step S1002. As a result of the comparison, if the similarity is equal to or greater than the similarity threshold (step S1003; Yes), the process proceeds to step S1004. If the similarity is less than the similarity threshold (step S1003; No), the process does not proceed to step S1004, and the looping of steps S1002 to S1004 is ended.

Step S1004 is a process in which the analysis target selection unit 223 records true in the condition column of the determination result table.

After the looping of step S1002 to step S1004 is executed for all the columns, the analysis target selection unit 223 proceeds to step S1005.

Step S1005 is a step in which the analysis target selection unit 223 determines whether all the columns of the determination result table are true. If all the columns are true (step S1005; Yes), the process proceeds to step S1006. If there is a column that is not true (step S1005; No), the process ends without proceeding to step S1006.

Step S1006 is a step in which the analysis target selection unit 223 acquires all records in the skeleton coordinate table 232 and stores the records in the analysis target table 251. After step S1006, the analysis target selection process ends.

According to the first embodiment, the measurement apparatus 110 selects the measurement data based on the similarities of the measurement conditions obtained from the horizontal plane, the traveling direction, and the camera coordinate system, thereby acquiring data close to ideal measurement data obtained by imaging under the measurement conditions assumed by the system. For example, even when the measurement apparatus 110 is installed at a plurality of different locations and the measurement data cannot be acquired under completely the same condition due to the restriction of the imaging location or the like, data measured under conditions similar to assumed conditions can be efficiently acquired. For this reason, it is possible to implement highly reliable analysis while considering the restriction of the imaging location, without collecting a large amount of data.

### Second Embodiment

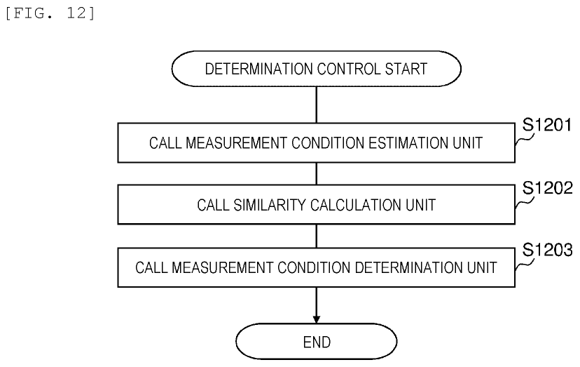

In a second embodiment, a system that determines a measurement condition will be described. FIG. 11 is a logical configuration diagram of the system according to the second embodiment. The measurement apparatus 110 in the system illustrated in FIG. 11 further includes a measurement condition determination unit 224, a determination control unit 225, and a U/I control unit 260 in addition to the configuration of the first embodiment. Other configurations and operations are similar to those of the first embodiment, and the same components are denoted by the same reference signs and a description thereof is omitted. Although not illustrated, the measurement DB 230 and the determination DB 240 store data similar to that in the first embodiment. Further, although not illustrated, the measurement apparatus 110 may be connected to the analysis apparatus 100 similarly to the first embodiment.

The measurement condition determination unit 224 is one of functions of the determination unit 220, and determines a measurement condition based on a similarity calculated by the similarity calculation unit 222.

The determination control unit 225 is one of the functions of the determination unit 220, and controls all processes related to the determination of the measurement condition.

The U/I control unit 260 is connected to the input device 115 and the output device 116, and controls input and output related to a measurement conditions.

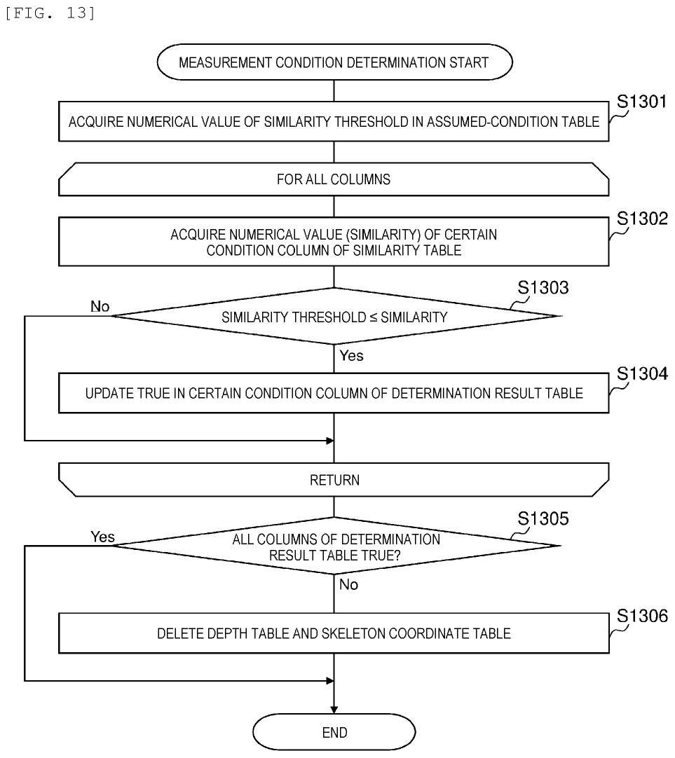

FIG. 12 is a flowchart illustrating a flow of a determination control process. When the process is started, the determination control unit 225 sequentially executes processes of step S1201 to step S1203.

Step S1201 is a step in which the determination control unit 225 calls the measurement condition estimation unit 221. Similarly to the first embodiment, the measurement condition estimation unit 221 estimates a measurement condition and stores the measurement condition in the measurement condition table 241.

Step S1202 is a step in which the determination control unit 225 calls the similarity calculation unit 222. Similarly to the first embodiment, the similarity calculation unit 222 calculates a similarity and stores the similarity in the similarity table 243.

Step S1203 is a step in which the determination control unit 225 calls the measurement condition determination unit 224. After the processes performed by the determination control unit 225, the determination control process ends.

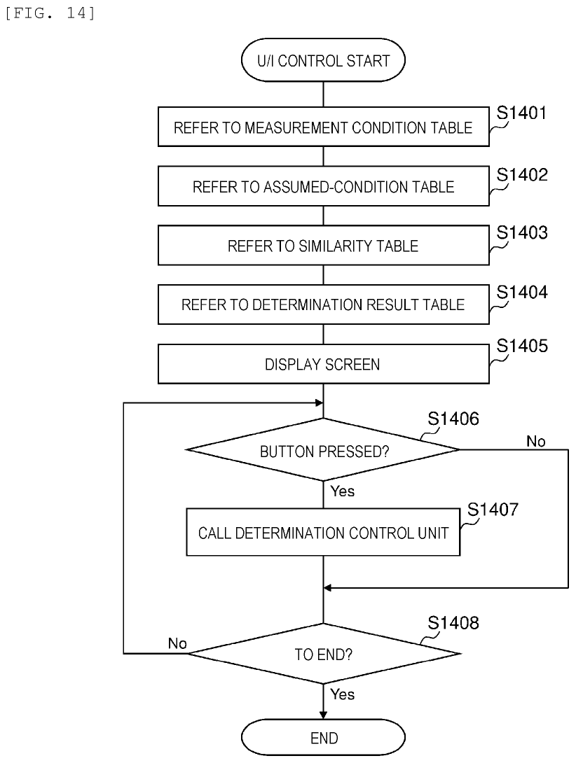

FIG. 13 is a flowchart illustrating a flow of a measurement condition determination process. When called by the determination control unit 225, the measurement condition determination unit 224 first executes a next step S1301.

Step S1301 is a step in which the measurement condition determination unit 224 acquires a numerical value of the similarity threshold in the assumed-condition table 242. Thereafter, the measurement condition determination unit 224 executes looping of step S1302 to step S1304 for all columns.

Step S1302 is a step in which the measurement condition determination unit 224 acquires a numerical value (similarity) of a certain condition column of the similarity table.

Step S1303 is a step in which the measurement condition determination unit 224 compares the similarity threshold acquired in step S1301 with the similarity acquired in step S1302. As a result of the comparison, if the similarity is equal to or greater than the similarity threshold (step S1303; Yes), the process proceeds to step S1304. If the similarity is less than the similarity threshold (step S1303; No), the process does not proceed to step S1304, and the looping of step S1302 to step S1304 is ended.

Step S1304 is a process in which the measurement condition determination unit 224 records true in the condition column of the determination result table.

After the looping of step S1302 to step S1304 is executed for all the columns, the measurement condition determination unit 224 proceeds to step S1305.

Step S1305 is a step in which the measurement condition determination unit 224 determines whether all the columns of the determination result table are true. If there is a column that is not true (step S1305; No), the process proceeds to step S1306. If all the columns are true (step S1305; Yes), the process ends without proceeding to step S1306.

Step S1306 is a step in which the measurement condition determination unit 224 deletes the depth table 231 and the skeleton coordinate table 232. After step S1306, the measurement condition determination process ends.

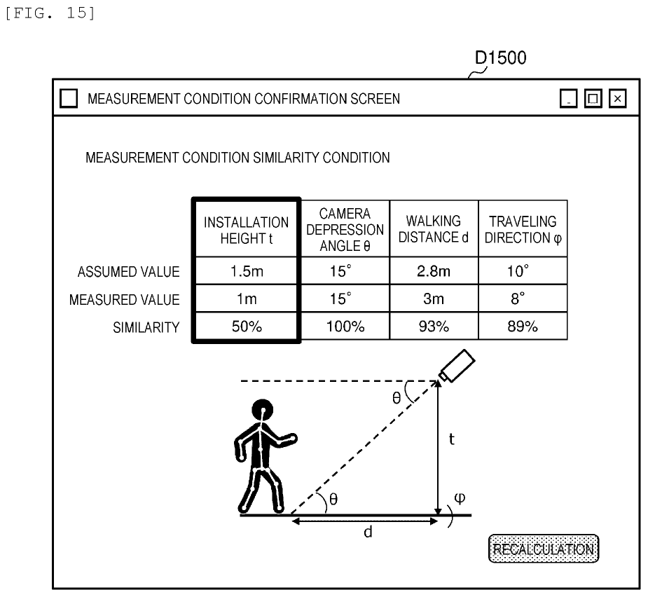

FIG. 14 is a flowchart illustrating a flow of a U/I control process. The U/I control unit 260 refers to the measurement condition table 241 (step S1401), refers to the assumed-condition table 242 (step S1402), refers to the similarity table 243 (step S1403), and refers to the determination result table 244 (step S1404).

Thereafter, the U/I control unit 260 causes the output device 116 to display a measurement condition confirmation screen indicating the measurement condition, the assumed condition, the similarity, and the determination result (step S1405). After step S1405, the U/I control unit 260 determines whether an operation for requesting re-execution of measurement condition determination is received (step S1406). When the re-execution operation of the measurement condition determination is received (step S1406; Yes), the U/I control unit 260 calls the determination control unit 225 (step S1407).

After step S1407 or when no re-execution operation of the measurement condition determination is received (step S1406; No), the U/I control unit 260 determines whether to end the display of the screen (step S1408). When the display of the screen is not to be ended (step S1408; No), the process returns to step S1406. When the display of the screen is to be ended (step S1408; Yes), the U/I control is ended together with the end of the display of the screen.

FIG. 15 is an explanatory diagram illustrating an example of a configuration of the measurement condition confirmation screen. A measurement condition confirmation screen D1500 illustrated in FIG. 15 comparatively displays an assumed value, a measured value, and a similarity for each item of the installation height, the camera depression angle, the walking distance, and the traveling direction. Regarding the item of the installation height of which the similarity is lower than the similarity threshold, it is indicated, by making a display mode of the item different from those of the other items, that the item is an item to be improved.

Further, the measurement condition confirmation screen D1500 is provided with a “recalculation” button for generating and displaying a design indicating a positional relationship between a target and the imaging device 200 based on the installation height, the camera depression angle, the walking distance, and the traveling direction, and requesting re-execution of the measurement condition determination.

From the measurement condition confirmation screen D1500, an operator can recognize that it is necessary to improve the installation height. When the installation height of the imaging device 200 is changed, imaging is performed again, and the recalculation button is operated, an evaluation result of the measurement condition after the change is displayed. By repeating this operation, the measurement condition can be brought close to the assumed condition.

According to the second embodiment, the measurement apparatus 110 can select and correct the measurement condition according to the restriction of the imaging location by using the similarity of the measurement condition. Accordingly, it is possible to implement highly reliable analysis while considering the restriction of the imaging location, without collecting a large amount of data.

As described above, the measurement system disclosed in the embodiments includes: the sensor 117 serving as a measurement data acquisition unit configured to acquire measurement data of a target; and the processor 111 serving as a processing unit configured to process the measurement data. The processing unit estimates a skeleton of a living body that is the target based on the measurement data, estimates a measurement condition related to the measurement data using an estimation result of the skeleton, obtains a similarity between the measurement condition and an assumed condition assumed in advance, and selects measurement data related to evaluation of a state of the target based on the similarity.

With the configuration and operations, the measurement system can efficiently collect the data related to the evaluation of the state of the target while considering a restriction of an imaging location without collecting a large amount of data.

As an example, the measurement data is data obtained by measuring walking of the target in a time series. In this case, the measurement condition may include a traveling direction and/or a distance of the walking.

With the configuration and operations, it is possible to collect the data for evaluating the state of the target from a state of walking, that is, a walking pattern.

As an example, the measurement data includes imaging data obtained by imaging the target with a camera, and the measurement condition includes an installation height and/or an imaging direction of the camera. The measurement data may further include depth data associated with the imaging data.

With the configuration and operations, it is possible to efficiently collect data with similar imaging conditions of the camera.

In the measurement system of the disclosure, the measurement condition and the assumed condition may be comparatively displayed. Therefore, it is possible to easily correct the measurement condition and bring the measurement condition close to the assumed condition.

As an example, the processing unit obtains the similarity by using a value obtained by normalizing a difference between the measurement condition and the assumed condition. As an example, the processing unit performs the selection by comparing the similarity with a similarity threshold. Therefore, it is possible to easily evaluate how similar the measurement condition is to the assumed condition, and to select the measurement data obtained under the measurement condition similar to the assumed condition.

As an example, the processing unit selects data related to evaluation of a health state of the target. The selected data may be used for training of a machine learning model and/or evaluation of the health state by the machine learning model. This configuration contributes to quantifying a health index of the target.

Although several embodiments of the invention have been described above, the embodiments are only examples for describing the invention, and the scope of the invention is not limited to these embodiments. The invention can be implemented in various other forms.

For example, although the configuration in which the measurement data for which the similarities of all the items are equal to or greater than the similarity threshold is selected is illustrated in each of the embodiments described above, a plurality of items may be combined to obtain one evaluation value and the selection may be performed based on the evaluation value.

Although a case where the configuration for performing evaluation of the measurement condition is added to the configuration for performing selection of the measurement data is illustrated in the second embodiment described above, a configuration for performing only the evaluation of the measurement condition may be implemented.

Although the configuration in which the measurement condition confirmation screen is output for the operator of the measurement apparatus 110 is illustrated in the second embodiment, a configuration may be implemented in which the measurement condition confirmation screen is output for an operator of the analysis apparatus 100 and the operator of the analysis apparatus 100 instructs the operator of the measurement apparatus 110 to improve the measurement condition.

Although the sensor 117 is described as a specific example of the measurement data acquisition unit in each of the embodiments described above, the measurement data acquisition unit is not necessarily a sensor. For example, it is possible to adopt a configuration in which the measurement data is acquired by any method such as reading measured data or receiving measured data from another device.

### REFERENCE SIGNS LIST

100: analysis apparatus
101: processor
102: memory
103: storage device
104: network interface
110: measurement apparatus
111: processor
112: memory
113: storage device
114: network interface
115: input device
116: output device
117: sensor
120: network
200: imaging device
210: measurement unit
211: depth recording unit
212: skeleton coordinate estimation unit
220: determination unit
221: measurement condition estimation unit
222: similarity calculation unit
223: analysis target selection unit
224: measurement condition determination unit
225: determination control unit
230: measurement DB
231: depth table
232: skeleton coordinate table
240: determination DB
241: measurement condition table
242: assumed-condition table
243: similarity table
244: determination result table
250: analysis DB
251: analysis target table
260: U/I control unit

1. A measurement system comprising:
a measurement data acquisition unit configured to acquire measurement data of a target; and
a processing unit configured to process the measurement data, wherein
the processing unit
estimates a skeleton of a living body that is the target based on the measurement data,
estimates a measurement condition related to the measurement data using an estimation result of the skeleton,
obtains a similarity between the measurement condition and an assumed condition assumed in advance, and
selects measurement data related to evaluation of a state of the target based on the similarity.

2. The measurement system according to claim 1, wherein
the measurement data is data obtained by measuring walking of the target in a time series.

3. The measurement system according to claim 2, wherein
the measurement condition includes a traveling direction and/or a distance of the walking.

4. The measurement system according to claim 1, wherein
the measurement data includes imaging data obtained by imaging the target with a camera, and
the measurement condition includes an installation height and/or an imaging direction of the camera.

5. The measurement system according to claim 4, wherein
the measurement data further includes depth data associated with the imaging data.

6. The measurement system according to claim 1, further comprising:
an output control unit configured to comparatively display the measurement condition and the assumed condition.

7. The measurement system according to claim 1, wherein
the processing unit obtains the similarity by using a value obtained by normalizing a difference between the measurement condition and the assumed condition.

8. The measurement system according to claim 1, wherein
the processing unit performs the selection by comparing the similarity with a similarity threshold.

9. The measurement system according to claim 1, wherein
the processing unit selects measurement data related to evaluation of a health state of the target.

10. The measurement system according to claim 9, wherein
the selected data is used for training of a machine learning model and/or evaluation of the health state by the machine learning model.

11. A measurement apparatus comprising:
a measurement data acquisition unit configured to acquire measurement data of a target; and
a processing unit configured to process the measurement data, wherein
the processing unit
estimates a skeleton of a living body that is the target based on the measurement data,
estimates a measurement condition related to the measurement data using an estimation result of the skeleton,
obtains a similarity between the measurement condition and an assumed condition assumed in advance, and
selects measurement data related to evaluation of a state of the target based on the similarity.

12. A measurement data processing method comprising:
a measurement data acquisition step of acquiring measurement data of a target;
a skeleton estimation step of estimating a skeleton of a living body that is the target based on the measurement data;
a condition estimation step of estimating a measurement condition related to the measurement data using an estimation result of the skeleton;
a similarity calculation step of obtaining a similarity between the measurement condition and an assumed condition assumed in advance; and
a selecting step of selecting measurement data related to evaluation of a state of the target based on the similarity.

13. A measurement data processing program for causing a computer to execute:
a process of receiving measurement data of a target;
a process of estimating a skeleton of a living body that is the target based on the measurement data;
a process of estimating a measurement condition related to the measurement data using an estimation result of the skeleton;
a process of obtaining a similarity between the measurement condition and an assumed condition assumed in advance; and
a process of selecting measurement data related to evaluation of a state of the target based on the similarity.
